(19) United States
(12) Patent Application Publication
(10) Pub. No.: US 2014/0271591 A1
(43) Pub. Date: Sep. 18, 2014

(54) Compositions and methods for the modulation of hemoglobin (s)

(71) Applicant: Global Blood Therapeutics, Inc., South San Francisco, CA (US)

(72) Inventors: Uma Sinha, South San Francisco, CA (US); Brian W. Metcalf, South San Francisco, CA (US); Donna Oksenberg, South San Francisco, CA (US)

(73) Assignee: Global Blood Therapeutics, Inc., South San Francisco, CA (US)

(21) Appl. No.: 13/815,872

(22) Filed: Mar. 15, 2013

(51) Int. Cl. A61K 31/444; A61K 35/18; C07D 401/04; A61K 31/4439; C07D 401/14; C07D 213/69

(52) U.S. Cl. CPC A61K 31/444; C07D 401/14; C07D 213/69; C07D 401/04; A61K 31/4439; A61K 35/18
USPC 424/93.73; 514/333; 514/335; 514/341

(57) ABSTRACT

This invention provides pharmaceutical compositions for the allosteric modulation of hemoglobin (S) and methods for their use in treating disorders mediated by hemoglobin (S) and disorders that would benefit from tissue and/or cellular oxygenation.

### FIELD OF THE INVENTION

This invention provides pharmaceutical compositions for the allosteric modulation of hemoglobin (S) and methods for their use in treating disorders mediated by hemoglobin (S) and disorders that would benefit from tissue and/or cellular oxygenation.

### STATE OF THE ART

Sickle cell disease is a disorder of the red blood cells, found particularly among those of African and Mediterranean descent. The basis for sickle cell disease is found in sickle hemoglobin (HbS or hemoglobin (S)), which contains a point mutation relative to the prevalent peptide sequence of hemoglobin (Hb).

Hemoglobin (Hb) transports oxygen molecules from the lungs to various tissues and organs throughout the body. Hemoglobin binds and releases oxygen through conformational changes. Sickle hemoglobin (HbS) contains a point mutation where glutamic acid is replaced with valine, allowing HbS to become susceptible to polymerization to give the HbS containing red blood cells having their characteristic sickle shape. The sickled cells are also more rigid than normal red blood cells, and their lack of flexibility can lead to blockage of blood vessels. U.S. Pat. No. 7,160,910 discloses compounds that are allosteric modulators of hemoglobin. However, a need exists for additional therapeutics that can treat disorders that are mediated by Hb or by abnormal Hb such as HbS.

### SUMMARY OF THE INVENTION

This invention relates generally to compositions suitable as allosteric modulators of hemoglobin (S). In some aspects, this invention relates to methods for treating disorders mediated by hemoglobin (S) and disorders that would benefit from tissue and/or cellular oxygenation.

In further aspects, this invention relates to a pharmaceutical composition comprising from about 1 mg to about 10 g of a compound selected from the group consisting of a compound in Table 1 and at least a pharmaceutically acceptable excipient, carrier or diluent.

In still further aspects, this invention relates to a blood composition comprising blood and one or more compounds selected from the group consisting of a compound in Table 1, wherein said blood is comprised of red blood cells and plasma, and wherein at least 20%, preferably at least 30%, more preferably at least 50%, yet more preferably at least 80%, and still more preferably at least 90% of said one or more compounds in the blood will bind to red blood cells containing hemoglobin (S) under physiological conditions.

In further aspects of this invention, a blood composition is provided wherein said composition comprises a compound in Table 1 and blood, said blood comprising red blood cells comprising hemoglobin, at least a part of the hemoglobin being hemoglobin (S), and at least a part of said hemoglobin (S) is present as an adduct with said compound.

In another aspect of the invention, provided herein are adducts of hemoglobin (S) and a compound selected from the group consisting of a compound in Table 1.

In a preferred embodiment, a compound selected from the group consisting of Table 1, present in the adduct of the red blood cells and Hb-S, has a volume of distribution between the vascular space and the extra-vascular space under steady state conditions such that at least a portion of the compound remains in the vascular space as part of said adduct. In one aspect, at least 20%, preferably at least 40%, yet more preferably at least 60%, still more preferably at least 80% and even more preferably at least 95% of said compound remains in the vascular space as part of said adduct.

In a further aspect, the invention relates to a method for treating a subject in need thereof, comprising administering to the subject an effective amount of a pharmaceutical composition according to the invention.

These and other aspects of the invention are further described below.

### DETAILED DESCRIPTION OF THE INVENTION

### Definitions

It must be noted that as used herein and in the appended claims, the singular forms “a”, “an”, and “the” include plural referents unless the context clearly dictates otherwise. Thus, for example, reference to “a solvent” includes a plurality of such solvents.

As used herein, the term “comprising” or “comprises” is intended to mean that the compositions and methods include the recited elements, but not excluding others. “Consisting essentially of” when used to define compositions and methods, shall mean excluding other elements of any essential significance to the combination for the stated purpose. Thus, a composition or process consisting essentially of the elements as defined herein would not exclude other materials or steps that do not materially affect the basic and novel characteristic(s) of the claimed invention. “Consisting of” shall mean excluding more than trace elements of other ingredients and substantial method steps. Embodiments defined by each of these transition terms are within the scope of this invention.

Unless otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations. Each numerical parameter should at least be construed in light of the number of reported significant digits and by applying ordinary rounding techniques. The term “about” when used before a numerical designation, e.g., temperature, time, amount, and concentration, including range, indicates approximations which may vary by (+) or (−) 10%, 5% or 1%.

The term “pharmaceutically acceptable” refers to safe and non-toxic for in vivo, preferably, human administration.

The term “pharmaceutically acceptable salt” refers to a salt that is pharmaceutically acceptable.

The term “salt” refers to an ionic compound formed between an acid and a base. When the compound provided herein contains an acidic functionality, such salts include, without limitation, alkali metal, alkaline earth metal, and ammonium salts. As used herein, ammonium salts include, salts containing protonated nitrogen bases and alkylated nitrogen bases. Exemplary, and non-limiting cations useful in pharmaceutically acceptable salts include Na, K, Rb, Cs, NH₄, Ca, Ba, imidazolium, and ammonium cations based on naturally occurring amino acids. When the compounds utilized herein contain basic functionality, such salts include, without limitation, salts of organic acids, such as caroboxylic acids and sulfonic acids, and mineral acids, such as hydrogen halides, sulfuric acid, phosphoric acid, and the likes. Exemplary and non-limiting anions useful in pharmaceutically acceptable salts include oxalate, maleate, acetate, propionate, succinate, tartrate, chloride, sulfate, bisalfate, mono-, di-, and tribasic phosphate, mesylate, tosylate, and the likes.

The term “whole blood” refers to blood containing all its natural constituents, components, or elements or a substantial amount of the natural constituents, components, or elements. For example, it is envisioned that some components may be removed by the purification process before administering the blood to a subject.

The terms “treat”, “treating” or “treatment”, as used herein, include alleviating, abating or ameliorating a disease or condition or one or more symptoms thereof, preventing additional symptoms, ameliorating or preventing the underlying metabolic causes of symptoms, inhibiting the disease or condition, e.g., arresting or suppressing the development of the disease or condition, relieving the disease or condition, causing regression of the disease or condition, relieving a condition caused by the disease or condition, or suppressing the symptoms of the disease or condition, and are intended to include prophylaxis. The terms also include relieving the disease or conditions, e.g., causing the regression of clinical symptoms. The terms further include achieving a therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the individual, notwithstanding that the individual is still be afflicted with the underlying disorder. For prophylactic benefit, the compositions are administered to an individual at risk of developing a particular disease, or to an individual reporting one or more of the physiological symptoms of a disease, even though a diagnosis of this disease has not been made.

The terms “preventing” or “prevention” refer to a reduction in risk of acquiring a disease or disorder (i.e., causing at least one of the clinical symptoms of the disease not to develop in a subject that may be exposed to or predisposed to the disease but does not yet experience or display symptoms of the disease). The terms further include causing the clinical symptoms not to develop, for example in a subject at risk of suffering from such a disease or disorder, thereby substantially averting onset of the disease or disorder.

The term “effective amount” refers to an amount that is effective for the treatment of a condition or disorder by an intranasal administration of a compound or composition described herein. In some embodiments, an effective amount of any of the compositions or dosage forms described herein is the amount used to treat a disorder mediated by hemoglobin or a disorder that would benefit from tissue and/or cellular oxygenation of any of the compositions or dosage forms described herein to a subject in need thereof.

The term “carrier” as used herein, refers to relatively nontoxic chemical compounds or agents that facilitate the incorporation of a compound into cells, e.g., red blood cells, or tissues.

### Compounds

A compound utilized herein is selected from Table 1 below or an N-oxide thereof, or a pharmaceutically acceptable salt of each thereof. The N-oxides of the compounds set forth below are believed to be novel and each of the N-oxide compounds and their salts thereof form a further embodiment of the invention.

The compounds in Table 1 represent compounds capable of meeting one or more biological criteria for activity as measured based on one or more biological parameters such as, but not limited to, partitioning between red blood cells and blood plasma, volume of distribution, oxygen equilibrium curves, oxygen affinity and polymerization activity.

TABLE 1

| Compound | | |
|---|---|---|
| Number | Chemical Structure | Chemical Name |
| 1 | [chemical structure] | 2-methoxy-5-[[2-(1H-pyrazol-5-yl)pyridin-3- yl]methoxy]pyridine-4-carbaldehyde |
| 2 | [chemical structure] | 2-methoxy-5-[[5-(2-methylpyrazol-3-yl)pyridin- 3-yl]methoxy]pyridine-4-carbaldehyde |
| 3 | [chemical structure] | 2-methoxy-5-[[2-(1-methylpyrazol-3-yl)pyridin- 3-yl]methoxy]pyridine-4-carbaldehyde |
| 4 | [chemical structure] | 2-methoxy-5-[[2-(2H-tetrazol-5-yl)pyridin-3- yl]methoxy]pyridine-4-carbaldehyde |
| 5 | [chemical structure] | 2-methoxy-5-[[2-(4-methyl-1H-pyrazol-5- yl)pyridin-3-yl]methoxy]pyridine-4-carbaldehyde |
| 6 | [chemical structure] | 2-methoxy-5-[(2-pyrazol-1-ylpyridin-3- yl)methoxy]pyridine-4-carbaldehyde |
| 7 | [chemical structure] | 5-[[2-(1,5-dimethylpyrazol-4-yl)pyridin-3- yl]methoxy]-2-methoxypyridine-4-carbaldehyde |
| 8 | [chemical structure] | 5-[[2-(2-ethylpyrazol-3-yl)pyridin-3-yl]methoxy]- 2-methoxypyridine-4-carbaldehyde |
| 9 | [chemical structure] | 2-methoxy-5-[[2-(2-propan-2-ylpyrazol-3- yl)pyridin-3-yl]methoxy]pyridine-4-carbaldehyde |
| 10 | [chemical structure] | 2-methoxy-5-[(2-phenylpyridin-3- yl)methoxy]pyridine-4-carbaldehyde |
| 11 | [chemical structure] | 2-methoxy-5-[[3-(2-propan-2-ylpyrazol-3- yl)pyridin-4-yl]methoxy]pyridine-4-carbaldehyde |
| 12 | [chemical structure] | 2-hydroxy-6-[[2-(2-propan-2-ylpyrazol-3- yl)pyridin-3-yl]methoxy]benzaldehyde |
| 13 | [chemical structure] | 2-methoxy-5-[(2-pyridin-3-ylpyridin-3- yl)methoxy]pyridine-4-carbaldehyde |
| 14 | [chemical structure] | 2-methoxy-5-[[2-[2-(2-methoxyethyl)pyrazol-3- yl]pyridin-3-yl]methoxy]pyridine-4-carbaldehyde |
| 15 | [chemical structure] | 5-[[2-[2-(2-hydroxyethyl)pyrazol-3-yl]pyridin-3- yl]methoxy]-2-methoxypyridine-4-carbaldehyde |
| 16 | [chemical structure] | 2-methoxy-5-[[2-(2-propylpyrazol-3-yl)pyridin-3- yl]methoxy]pyridine-4-carbaldehyde |
| 17 | [chemical structure] | 2-methoxy-5-[[2-[2-(2,2,2-trifluoroethyl)pyrazol- 3-yl]pyridin-3-yl]methoxy]pyridine-4- carbaldehyde |
| 18 | [chemical structure] | 5-[[2-(2-cyclobutylpyrazol-3-yl)pyridin-3- yl]methoxy]-2-methoxypyridine-4-carbaldehyde |
| 19 | [chemical structure] | 5-[[2-(2-cyclohexylpyrazol-3-yl)pyridin-3- yl]methoxy]-2-methoxypyridine-4-carbaldehyde |
| 20 | [chemical structure] | 5-[[2-[2-(cyclohexylmethyl)pyrazol-3-yl]pyridin- 3-yl]methoxy]-2-methoxypyridine-4- carbaldehyde |
| 21 | [chemical structure] | 5-[[2-(2-cyclopentylpyrazol-3-yl)pyridin-3- yl]methoxy]-2-methoxypyridine-4-carbaldehyde |
| 22 | [chemical structure] | 5-[[2-[2-(2,2-difluoroethyl)pyrazol-3-yl]pyridin- 3-yl]methoxy]-2-methoxypyridine-4- carbaldehyde |
| 23 | [chemical structure] | 2-methoxy-5-[[2-(2-methylphenyl)pyridin-3- yl]methoxy]pyridine-4-carbaldehyde |
| 24 | [chemical structure] | 2-methoxy-5-[[2-(2-methoxypyridin-3-yl)pyridin- 3-yl]methoxy]pyridine-4-carbaldehyde |
| 25 | [chemical structure] | 2-methoxy-5-[[3-(2-propan-2-ylpyrazol-3- yl)pyrazin-2-yl]methoxy]pyridine-4-carbaldehyde |
| 26 | [chemical structure] | 2-(difluoromethoxy)-5-[[2-(2-propan-2-ylpyrazol- 3-yl)pyridin-3-yl]methoxy]pyridine-4- carbaldehyde |
| 27 | [chemical structure] | 2-(2-methoxyethoxy)-5-[[2-(2-propan-2- ylpyrazol-3-yl)pyridin-3-yl]methoxy]pyridine-4- carbaldehyde |
| 28 | [chemical structure] | 2-[5-[3-[(4-formyl-6-methoxypyridin-3- yl)oxymethyl]pyridin-2-yl]pyrazol-1-yl]acetic acid |
| 29 | [chemical structure] | 3-[[2-(2-propan-2-ylpyrazol-3-yl)pyridin-3- yl]methoxy]pyridine-2-carbaldehyde |
| 30 | [chemical structure] | 6-methyl-3-[[2-(2-propan-2-ylpyrazol-3- yl)pyridin-3-yl]methoxy]pyridine-2-carbaldehyde |
| 31 | [chemical structure] | 5-[[2-(2-hydroxypropan-2-yl)pyridin-3- yl]methoxy]-2-methoxypyridine-4-carbaldehyde |
| 32 | [chemical structure] | 2-(2-methoxyethoxy)-5-[[2-(2-methylpyrazol-3- yl)pyridin-3-yl]methoxy]pyridine-4-carbaldehyde |
| 33 | [chemical structure] | methyl 3-[5-[3-[(4-formyl-6-methoxypyridin-3- yl)oxymethyl]pyridin-2-yl]pyrazol-1- yl]propanoate |
| 34 | [chemical structure] | 3-[5-[3-[(4-formyl-6-methoxypyridin-3- yl)oxymethyl]pyridin-2-yl]pyrazol-1-yl]propanoic acid |
| 35 | [chemical structure] | 3-hydroxy-5-[[2-(2-propan-2-ylpyrazol-3- yl)pyridin-3-yl]methoxy]pyridine-4-carbaldehyde |
| 36 | [chemical structure] | 3-methoxy-5-[[2-(2-propan-2-ylpyrazol-3- yl)pyridin-3-yl]methoxy]pyridine-4-carbaldehyde |
| 37 | [chemical structure] | 2-methoxy-5-[[2-(4-methyl-2-propan-2-ylpyrazol- 3-yl)pyridin-3-yl]methoxy]pyridine-4- carbaldehyde |
| 38 | [chemical structure] | 2-hydroxy-6-[[2-[2-(2,2,2-trifluoroethyl)pyrazol- 3-yl]pyridin-3-yl]methoxy]benzaldehyde |
| 39 | [chemical structure] | 2-hydroxy-6-[[2-[2-(3,3,3- trifluoropropyl)pyrazol-3-yl]pyridin-3- yl]methoxy]benzaldehyde |
| 40 | [chemical structure] | 2-(2-methoxyethoxy)-5-[[2-[2-(2,2,2- trifluoroethyl)pyrazol-3-yl]pyridin-3- yl]methoxy]pyridine-4-carbaldehyde |
| 41 | [chemical structure] | 2-methoxy-5-[[2-[2-(3,3,3- trifluoropropyl)pyrazol-3-yl]pyridin-3- yl]methoxy]pyridine-4-carbaldehyde |
| 42 | [chemical structure] | 2-(2-methoxyethoxy)-5-[[2-[2-(3,3,3- trifluoropropyl)pyrazol-3-yl]pyridin-3- yl]methoxy]pyridine-4-carbaldehyde |
| 43 | [chemical structure] | 6-methyl-3-[[2-[2-(2,2,2-trifluoroethyl)pyrazol-3- yl]pyridin-3-yl]methoxy]pyridine-2-carbaldehyde |
| 44 | [chemical structure] | 6-methyl-3-[[2-[2-(3,3,3-trifluoropropyl)pyrazol- 3-yl]pyridin-3-yl]methoxy]pyridine-2- carbaldehyde |
| 45 | [chemical structure] | 2-fluoro-6-[[2-[2-(2,2,2-trifluoroethyl)pyrazol-3- yl]pyridin-3-yl]methoxy]benzaldehyde |
| 46 | [chemical structure] | 2-fluoro-6-[[2-[2-(3,3,3-trifluoropropyl)pyrazol- 3-yl]pyridin-3-yl]methoxy]benzaldehyde |
| 47 | [chemical structure] | 3-[[2-[2-(2,2,2-trifluoroethyl)pyrazol-3- yl]pyridin-3-yl]methoxy]pyridine-2-carbaldehyde |
| 48 | [chemical structure] | 3-[[2-[2-(3,3,3-trifluoropropyl)pyrazol-3- yl]pyridin-3-yl]methoxy]pyridine-2-carbaldehyde |
| 49 | [chemical structure] | 3-chloro-5-[[2-(2-propan-2-ylpyrazol-3- yl)pyridin-3-yl]methoxy]pyridine-4-carbaldehyde |
| 50 | [chemical structure] | 2-fluoro-6-[[2-(2-propan-2-ylpyrazol-3- yl)pyridin-3-yl]methoxy]benzaldehyde |
| 51 | [chemical structure] | 3-methyl-5-[[2-(2-propan-2-ylpyrazol-3- yl)pyridin-3-yl]methoxy]pyridine-4-carbaldehyde |
| 52 | [chemical structure] | 3-methyl-5-[[2-[2-(2,2,2-trifluoroethyl)pyrazol-3- yl]pyridin-3-yl]methoxy]pyridine-4-carbaldehyde |

In a preferred embodiment, the compound is compound 12.

### Pharmaceutical Compositions

In further aspects of the invention, a composition is provided comprising any of the compounds described herein, and at least a pharmaceutically acceptable excipient wherein the compound of Table 1 is present in the composition in an amount from 1 mg to 10 g.

In another aspect, this invention provides a composition comprising any of the compounds described herein, and a pharmaceutically acceptable excipient.

Such compositions can be formulated for different routes of administration. Although compositions suitable for oral delivery will probably be used most frequently, other routes that may be used include transdermal, intravenous, intraarterial, pulmonary, rectal, nasal, vaginal, lingual, intramuscular, intraperitoneal, intracutaneous, intracranial, and subcutaneous routes. Suitable dosage forms for administering any of the compounds described herein include tablets, capsules, pills, powders, aerosols, suppositories, parenterals, and oral liquids, including suspensions, solutions and emulsions. Sustained release dosage forms may also be used, for example, in a transdermal patch form. All dosage forms may be prepared using methods that are standard in the art (see e.g., Remington's Pharmaceutical Sciences, 16th ed., A. Oslo editor, Easton Pa. 1980).

Pharmaceutically acceptable excipients are non-toxic, aid administration, and do not adversely affect the therapeutic benefit of the compound of this invention. Such excipients may be any solid, liquid, semi-solid or, in the case of an aerosol composition, gaseous excipient that is generally available to one of skill in the art. Pharmaceutical compositions in accordance with the invention are prepared by conventional means using methods known in the art.

The compositions disclosed herein may be used in conjunction with any of the vehicles and excipients commonly employed in pharmaceutical preparations, e.g., talc, gum arabic, lactose, starch, magnesium stearate, cocoa butter, aqueous or non-aqueous solvents, oils, paraffin derivatives, glycols, etc. Coloring and flavoring agents may also be added to preparations, particularly to those for oral administration. Solutions can be prepared using water or physiologically compatible organic solvents such as ethanol, 1,2-propylene glycol, polyglycols, dimethylsulfoxide, fatty alcohols, triglycerides, partial esters of glycerin and the like.

Solid pharmaceutical excipients include starch, cellulose, hydroxypropyl cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. In certain embodiments, the compositions provided herein comprises one or more of α-tocopherol, gum arabic, and/or hydroxypropyl cellulose.

In one embodiment, this invention provides sustained release formulations such as drug depots or patches comprising an effective amount of a compound provided herein. In another embodiment, the patch further comprises gum Arabic or hydroxypropyl cellulose separately or in combination, in the presence of alpha-tocopherol. Preferably, the hydroxypropyl cellulose has an average MW of from 10,000 to 100,000. In a more preferred embodiment, the hydroxypropyl cellulose has an average MW of from 5,000 to 50,000.

Compounds and pharmaceutical compositions of this invention may be used alone or in combination with other compounds. When administered with another agent, the co-administration can be in any manner in which the pharmacological effects of both are manifest in the patient at the same time. Thus, co-administration does not require that a single pharmaceutical composition, the same dosage form, or even the same route of administration be used for administration of both the compound of this invention and the other agent or that the two agents be administered at precisely the same time. However, co-administration will be accomplished most conveniently by the same dosage form and the same route of administration, at substantially the same time. Obviously, such administration most advantageously proceeds by delivering both active ingredients simultaneously in a novel pharmaceutical composition in accordance with the present invention.

In further aspects of the invention, one or more adducts of a compound selected from Table 1 that is bound to hemoglobin S is contemplated. In one embodiment, the adduct is formed from compound 12 and hemoglobin S.

### Methods of Treatment

This invention provides a method for increasing the oxygen-carrying capacity of erythryocytes. In certain embodiments, the invention is related to a method of treating red blood cells or whole blood in vivo, in vitro, in situ or ex vivo with one or more compounds or pharmaceutical compositions of the invention by administering or contacting said one or more compound or pharmaceutical compositions with blood and especially blood containing hemoglobin (S).

In some embodiments, a method for ex vivo storage and/or use of the compounds and pharmaceutical compositions of the invention is contemplated in which the compounds and/or pharmaceutical compositions are combined with whole blood for use in procedures such as, but not limited to, autologous or non-autologous blood transfusions, coronary bypass surgery, and any extracorporeal procedure involving perfusion and/or reperfusion of blood to a subject. In certain embodiments, the compounds and/or pharmaceutical compositions may be combined with whiole blood for storage purposes.

In another of this method aspects, this invention is directed to a method for treating a subject in need thereof (e.g., sickle cell anemia) by administering to the subject an effective amount of a pharmaceutical composition of this invention. In one preferred aspect, the pharmaceutical composition comprises from 0.1 mg/kg to 1 g/kg per day, more preferably, 1 mg/kg/day to 100 mg/kg/day of a compound or compounds of Table 1.

In aspects of the invention, a method is provided for increasing oxygen affinity of hemoglobin S in a subject, the method comprising administering to a subject in need thereof a therapeutically effective amount of a pharmaceutical composition of this invention or a blood composition comprising one or more compounds of Table 1. In a preferred embodiment, the blood composition is free of hemoglobin (S).

In aspects of the invention, a method is provided for treating a condition associated with oxygen deficiency, the method comprising administering to a subject in need thereof a therapeutically effective amount of either the pharmaceutical or the blood composition described above.

In further aspects of the invention, a method is provided for treating oxygen deficiency associated with sickle cell anemia, the method comprising administering to a subject in need thereof a therapeutically effective amount of either the pharmaceutical or the blood composition described above.

Additionally, the compounds and pharmaceutical compositions of the invention can be added to whole blood or packed cells preferably at the time of storage or at the time of transfusion. In some embodiments, the compounds and pharmaceutical compositions may be added to whole blood or red blood cell fractions in a closed system using an appropriate reservoir in which the compound or pharmaceutical composition is placed prior to storage or which is present in the anticoagulating solution in the blood collecting bag.

### Synthetic Methods

The synthesis of the compounds of Table 1 are described in U.S. Patent Ser. Nos. 61/661,320 and 61/581,053, each of which is incorporated herein by reference in their entireties, for the sole purpose of describing the synthesis of these compounds.

### EXAMPLES

The following examples are given for the purpose of illustrating various embodiments of the invention. They are not meant to limit the invention in any fashion. One skilled in the art will appreciate that the invention is well adapted to carry out the objects and obtain the ends and advantages mentioned, as well any objects, ends and advantages inherent herein. The present examples (along with the methods described herein) are presently representative of preferred embodiments. They are exemplary, and are not intended as limitations on the scope of the invention. Variations and other uses which are encompassed within the spirit of the invention as defined by the scope of the claims will occur to those skilled in the art.

### Example 1

The compounds provided in the present invention are allosteric modulators of hemoglobin. As such, these compounds do not modulate red blood cells by themselves. Instead, the response of red blood cells to a concentration of hemoglobin is increased when compounds of formula I are present. Compounds of formula 1 are expected to have their effect on red blood cells by virtue of their ability to enhance the function of hemoglobin.

This experiment was established and used in order to assess the pharmacokinetic (PK) properties of the compounds.

Sample collection and data analysis: Rats (Sprague-Dawley, male, 8-12 weeks old) were dosed with one of three compounds corresponding to compound 12, compound 22 or compound 23. The rats received oral (10 mg/kg) or intravenous (1 mg/kg) doses of the compound. Rats were fasted overnight before the experiments and provided with food after the 2 hour sampling time point.

Blood samples were collected at different time points. Blood was anti-coagulated by 3.2% TSC (trisodium citrate) and a portion was separated into plasma fraction by centrifugation and removal of blood cells. Plasma and lysed blood samples were analyzed for drug concentration using LC-MS/MS. PK parameters were calculated by non compartmental analysis of the concentration-time profiles using WinNonLin software (Pharsight, Mountain View, Calif.). Apparent elimination half-life (t_{1/2}) values were calculated as ln(2)/k. Area under the concentration-time curve (AUC) values were estimated using the linear trapezoidal method. AUCₗₐₛₜ values were calculated from the dosing time to the last measurable concentration. AUC_{inf} values were calculated as the sum of the corresponding AUCₗₐₛₜ and the ratio of the last detectable concentration divided by k. Plasma clearance (Cl) is calculated from Dose/AUC_{inf}. Volume of distribution at steady state (Vₛₛ) is calculated from Mean Residence Time_{inf}×Clₛₛ. Maximum concentration (Cₘₐₓ) and time to Cₘₐₓ (Tₘₐₓ) was recorded as observed. The blood/plasma partitioning ratio was calculated at each experimental time point.

Results: Table 2 summarizes select PK parameters for the compounds listed below:

TABLE 2

| PK Parameter | 12 | 22 | 23 |
|---|---|---|---|
| Vₛₛ (L/kg) | 0.14 | 3.1 | 3.15 |
| Cl (ml/min/kg) | 0.11 | 9 | 13.7 |
| Bioavailability (%) | 68.8 | 6.6 | 1.8 |
| Blood/plasma (Ratio of peak concentration) | 21 | 5 | 1 |
| Blood/plasma (Ratio of exposure AUC_{INF}) | 55 | 33 | 4 |

The volume of distribution for compound 12 is 0.14 L/kg which indicates that it is not significantly distributed into extravascular space in rats (control normal Vz=0.1 L/kg). Higher Vₛₛ are observed for two related compounds (compound 22 and compound 23, 3.1 and 3.15, respectively), indicating that these compounds are more likely to distribute into the extravascular space and additional compartments than compound 12.

However, when the red blood cell compartment is considered, compound 12 unexpectedly partitions into blood to a far greater extent than compound 22 or compound 23. When the compounds are dosed orally, the relative proportion in blood (as compared to plasma) at peak concentration (Cmax) were much higher for compound 12 (21×-fold) than for compound 22 (5×-fold) or compound 23 (3×-fold). When the red blood cell/plasma ratio was measured at the peak concentration, compound 12 partitioned at a ratio of 70 to 1 into the erythrocytes attesting to its preferential partition into the compartment which contains the drug target hemoglobin. Supportive data was reported in an in vitro system measuring binding of compound 12 to hemoglobin and human serum albumin. In this functional assay, when both proteins are present in their respective physiologic ratio, compound 12 demonstrated preferentially, binding to hemoglobin.

Another surprising and unexpected observation was detected when overall exposure was tracked in animals dosed orally with compound 12. There was a 55-fold higher level of compound in blood than in plasma compared with blood/plasma ratios for compound 22 (5×-fold) or compound 23 (1×-fold).

The ability of compound 12 to partition preferentially in red blood cells has also been confirmed in mice treated intravenously. A ratio of blood/plasma of 15.4 (at peak in vivo concentration) and 30 (at overall exposure) was observed in mice. Analogous to the measurements in rats, the volume of distribution (Vss) was low in mice (0.10). Thus, compound 12 is not expected to distribute broadly distribute into extravascular space in mice.

In conclusion, the results shown in Table 2 demosntrate that the lack of compound 12 distribution into extravascular tissues (low Vss) combined with selective partitioning into the target compartment (red blood cells) provide a potential basis for reduced toxicity.

Accordingly, provided herein are blood compositions comprising one or more compounds selected from Table 1, and blood, wherein in the blood, at least 30% of the compound or compounds are bound to the red blood cells present in the blood.

### Example 2

Another series of assays were conducted in order to assess additional pharmacokinetic (PK) properties of the compounds from Example 1.

### Reverse Hemox Assay

Oxygen Equilibrium Curves (OEC) of whole blood before and after treatment with different concentrations of compounds 12, 22 and 23 were performed as follows using a HEMOX analyzer (TCS Scientific, New Hope, Pa.). Blood samples from homozygous sickle cell patients were obtained though the Hemoglobinopathy Center at Children's Hospital Oakland Research Institute (CHORD with Institutional Review Board approval. The hematocrit was adjusted to 20% using autologous plasma and the blood samples were incubated for 1 hour at 37° C. in absence or presence of compounds. 100 μl of these samples were added to 5 mL of Hemox buffer (30 mM TES, 130 mM NaCl, 5 mM KCl, pH=7.4) at 37° C. and then transferred to the Hemox sample chamber. The samples were saturated with oxygen by flushing with compressed air for 10 minutes. The samples were then flushed with pure nitrogen and the respective absorbances of oxy- and deoxy-Hb are recorded as a function of the solution pO2. The oxygen equilibrium data were then fitted to the Hill Model to obtain values for p50. The deoxygenation curves for both whole blood alone (control) and whole blood in the presence of the compound were collected with the TCS software.

Results: Table 3 below lists the delta p50% values where “+” indicates a delta p50% of between 0 and 29, “++” indicates a delta p50% of between 30 and 50, and “+++” indicates a delta p50% of 50 or greater. A positive delta p50 value corresponds to a left shifted curve and a lower p50 value relative to control, indicating that the compound acts to modulate Hb(S) to increase its affinity for oxygen.

### R/T Assay

A relaxed-to-tense transition assay (“R/T assay”) was used to determine the ability of compounds 12, 22 and 23 to mantain the high-oxygen affinity relaxed (R) state of hemoglobin under deoxygenated conditions. This ability can be expressed as a “delta R” value (i.e., the change in the time-period of the R state after hemoglobin is treated with a compound, as compared to the period without treatment with the compound). Delta R is the % R to remaining after the compounds treatment compared with no treatment (e.g. if R % without treatment is 8% while with treatment with a target compound is 48% R at 30 μM, then % R is 40% for that compound.

[symbol]

A mixture of HbS/A was purified from blood obtained from homozygous sickle cell patients though the Hemoglobinopathy Center at Children's Hospital Oakland Research Institute (CHORD with Institutional Review Board approval. HbS/A (at a final concentration of 3 μM) was incubated for 1 hr at 37° C. in presence or absence of compounds in 50 μM potassium phosphate buffer, pH=7.4 and 30 μM 2, 3 diphosphoglycerate (DPG) in 96 well plates in a final volume of 160 Compounds were added at different concentrations (3 μM to 100 μM final concentrations). Plates were covered with a Mylar film. After incubation was completed the Mylar cover was removed and the plates were placed in a Spectrostar Nano plate reader previously heated at 37° C. Five minutes later, N₂ (flow rate=20 L/min) was flowed through the spectrophotometer. Spectroscopic measurements (300 nm to 700 nm) were taken every 5 min for 2 hours. Data analysis was performed by using linear regression from the data retrieved for all wavelengths.

Results: Table 3 below lists the delta R values where “+” indicates a delta R of between 0 and 30, “++” indicates a delta R of between 30 and 50, and “+++” indicates a delta R of 50 or greater.

### Polymerization Assay

Polymerization assays are carried out in vitro using purified HBS exchanged into 1.8 M potassium phosphate buffer at pH 7.4. Using a slightly modified protocol (Antonini and Brunori, 1971), HbS is purified by the CRO VIRUSYS, from blood obtained from homozygous sickle cell patients through the Hemoglobinopathy Center at Children's Hospital Oakland Research Institute (CHORI) with Institutional Review Board approval. Compounds are prepared in 100% DMSO and a desired amount is added to 50 μM of purified HBS at a final DMSO concentration of 0.3%. Final potassium phosphate concentration is adjusted to 1.8 M using a combination of 2.5 M potassium phosphate stock solution and water at pH 7.4. The reaction mixture is incubated for an hour at 37° C. and then transferred into a 24-well plate for deoxygenation in a glove box containing 99.5% nitrogen and 0.5% oxygen. The 24-well plate is not covered and incubated at 4° C. on a plate cooler inside the glove box for one and a half hours. Fifty μL of the reaction mixture is transferred into a 96-well plate and the absorbance at 700 nm is measured every minute for one hour at 37° C. in a plate reader located inside the glove box. A plot of the absorbance against time is fitted using a Boltzman sigmoidal fit and the delay time (from zero to time at half Vmax) is measured. To compare and rank compounds, delay times are expressed as percent delay (% DT), which is defined as the difference in delay times for HBS/compound and HBS alone multiplied by 100 and divided by the delay time for HBS alone.

Results: Compounds listed below have been tested in the polymerization assay. Activity ranges are defined by the number of dagger (†) symbols indicated. † denotes activity ≧40% but ≦80%; †† denotes activity ≧80% but ≦120%; ††† denotes activity >120% but ≦140%; †††† denotes activity >160%.

TABLE 3

| In Vitro Assay Parameter/Unit | 12 | 22 | 23 |
|---|---|---|---|
| Reverse Hemox (1 mM)/ | 79.83 (+++) | 68.69 (+++) | 72.45 (+++) |
| (delta p50%) | | | |
| R-T (9 μM)/(delta R) | 65.45 (+++) | 31.02 (++) | 37.15 (++) |
| R-T (10 μM)/(delta R) | 62.75 (+++) | 36.25 (++) | 51.55 (+++) |
| Polymerization (75 μM)/ | 108.56 (††) | 90.22 (††) | 98.19 (††) |
| (% DT) | | | |

From the foregoing it will be appreciated that, although specific embodiments of the invention have been described herein for purposes of illustration, various modifications may be made without deviating from the spirit and scope of the invention.

Throughout the description of this invention, reference is made to various patent applications and publications, each of which are herein incorporated by reference in their entirety.

What is claimed is:

1. A composition comprising from about 1 mg to about 10 g of a compound selected from the group consisting of a compound in Table 1 and at least a pharmaceutically acceptable excipient, carrier or diluent.

2. The composition of claim 1, wherein the compound is compound 12 in Table 1.

3. A blood composition comprising blood and one or more compounds selected from the group consisting of a compound in Table 1,
wherein said blood is comprised of red blood cells and plasma, and
wherein at least 20% of said one or more compounds in the blood is bound to said red blood cells under physiological conditions.

4. The blood composition of claim 3, wherein at least 30% of said one or more compounds is bound to said red blood cells.

5. The blood composition of claim 3, wherein at least 50% of said one or more compounds is bound to said red blood cells.

6. The blood composition of claim 3, wherein at least 80% of said one or more compounds is bound to said red blood cells.

7. The blood composition of claim 3, wherein at least 90% of said one or more compounds is bound to said red blood cells.

8. The blood composition of claim 3, wherein said composition is compound 12 in Table 1.

9. The blood composition of claim 3, wherein at least a part of said red blood cells is sickled, and at least a part of said hemoglobin is bound to said compound.

10. The blood composition of claim 3, wherein said blood is free of or substantially free of hemoglobin.

11. A blood composition comprising an adduct formed from blood and one more or compounds selected from the group consisting of a compound in Table 1,
wherein said blood is comprised of red blood cells and plasma,
wherein said adduct is distributed under steady state conditions between a vascular space and an extra-vascular space in vivo, and
wherein at least a portion of said one or more compounds remains in said vascular space as part of said adduct.

12. The blood composition of claim 11, wherein at least 20% of said one or more compounds remains in said vascular space as part of said adduct.

13. The blood composition of claim 11, wherein at least 40% of said one or more compounds remains in said vascular space as part of said adduct.

14. The blood composition of claim 11, wherein at least 60% of said one or more compounds remains in said vascular space as part of said adduct.

15. The blood composition of claim 11, wherein at least 80% of said one or more compounds remains in said vascular space as part of said adduct.

16. The blood composition of claim 11, wherein at least 95% of said one or more compounds remains in said vascular space as part of said adduct.

17. The blood composition of claim 11, wherein said composition is compound 12 in Table 1.

18. A method for treating a subject in need thereof, comprising administering to the subject an effective amount of a pharmaceutical composition of claim 1.
